(19) United States
(12) Patent Application Publication
(10) Pub. No.: US 2024/0358299 A1
(43) Pub. Date: Oct. 31, 2024

(54) Blood Sample Collection System with Diversion Syringe Having an Integrated Scrubbing Cap

(71) Applicant: Becton, Dickinson and Company, Franklin Lakes, NJ (US)

(72) Inventor: Jonathan Karl Burkholz, Salt Lake City, UT (US)

(21) Appl. No.: 18/644,768

(22) Filed: Apr. 24, 2024

Related U.S. Application Data
(60) Provisional application No. 63/461,685, filed on Apr. 25, 2023.

(51) Int. Cl. A61B 5/15; A61M 39/10; A61M 39/16

(52) U.S. Cl. CPC A61B 5/150992; A61M 39/10; A61M 39/16; A61M 2039/1033

(57) ABSTRACT

Provided herein is a blood collection system including a syringe assembly and a container. The syringe assembly includes a syringe having a chamber and a fluid connector component, and a plunger assembly including an adapter member, a receiving cavity, and a fluid access component. The adapter member includes a stopper slidable within the barrel, and a channel extending between the stopper and an opening of the receiving cavity, with the fluid access component positioned over the opening and extending into the receiving cavity. A cannula is secured within the fluid connector component and extends proximally through the stopper to fluidly connect the fluid connector component with the channel. A scrubbing cap is secured to the fluid connector component that includes a housing defining a cavity within which a scrubbing insert is positioned. The container is positionable within the receiving cavity and may be engaged with the fluid access component.

### CROSS-REFERENCE TO RELATED APPLICATION

The present application claims priority to U.S. Provisional Application No. 63/461,685 entitled “Blood Sample Collection System with Diversion Syringe Having an Integrated Scrubbing Cap” filed Apr. 25, 2023, the entire disclosure of which is hereby incorporated by reference.

### BACKGROUND OF THE INVENTION

### Field of the Invention

The present disclosure relates to a blood collection system that includes a diversion syringe with an integrated scrubbing cap.

### Description of Related Art

Catheters are commonly used to administer fluids into and draw fluids (i.e., blood) out of the barrel, with patients in a variety of settings, including in hospitals and in home care, being administered such fluids or having blood drawn via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

In some applications, a VAD is used for collection of a blood specimen in connection with performing of a blood culture. Blood cultures are often used as a tool to detect the presence of bacteria or fungi in a blood sample of a patient, to identify the type of bacteria or fungi present, and to direct the treatment of the patient. The current practice of collecting a venous blood sample for blood culture testing involves a venipuncture with a blood collection set with an attached luer-lock access device (LLAD). During the insertion process, the venipuncture device may pick up microbes from the skin and insertion process and later transfer such microbes into the blood culture sample leading to a false positive test result for sepsis. To minimize the risk of a false positive, a discard sample is typically taken to remove the initial volume of blood that more likely contains microbes from the skin, such as discarding the first 0.1-10 ml of blood drawn.

In some prior art devices, a syringe integrated with an LLAD is provided that facilities in-line diversion of the initial blood volume by retracting the syringe plunger. Once the initial blood volume is diverted and isolated, the blood culture sample collection container is connected to the LLAD, allowing the blood culture sample to flow into the container. Although this addresses some of the risk of a false positive blood culture sample, it does not address the risk of a clinician failing to disinfect or ineffectively disinfecting the VAD access port prior to connecting the blood collection device thereto. That is, clinical practitioners often use alcohol wipes for scrubbing and disinfecting the access port before connecting a LLAD thereto, but use of alcohol wipes for scrubbing and disinfecting the access port is oftentimes not completely effective in disinfecting all internal/external surfaces of the access port, including (potentially) the intricate thread areas around the a luer portion thereof that couples with the LLAD. Additionally, it is possible in some instances for the clinician to forget to wipe down the access port prior to connection of the LLAD. If the disinfection of the access port is not performed properly, a false positive blood culture sample may arise.

Accordingly, a need exists in the art for a means for ensuring that a clinician effectively disinfects the access port of the VAD prior to attaching a blood collection device thereto.

### SUMMARY OF THE INVENTION

Provided herein is a blood collection system that includes a syringe assembly and a container. The syringe assembly includes a syringe having a distal end and a proximal end, with the syringe defining a chamber and having a fluid connector component at the distal end. The syringe assembly also includes a plunger assembly including an adapter member, a receiving cavity, and a fluid access component. The adapter member has a distal end including a stopper insertable into the proximal end of the syringe and slidable within the barrel, as well as a channel extending along a length thereof between the stopper and an opening of the receiving cavity. The fluid access component is positioned over the opening and extends into the receiving cavity. The syringe assembly further includes a cannula having a distal end and a proximal end, the distal end of the cannula secured within the fluid connector component, with the cannula extending proximally through the stopper to place the fluid connector component in fluid communication with the channel and the fluid access component. The syringe assembly still further includes a scrubbing cap secured to the fluid connector component of the syringe, the scrubbing cap including a housing secured to the fluid connector component and defining a cavity, and a scrubbing insert positioned within the cavity. The container defines a reservoir and is positionable within the receiving cavity and engageable with the fluid access component to transfer fluid into the reservoir.

In some embodiments, the scrubbing insert comprises a resilient material including an antimicrobial solution or agent absorbed therein configured to disinfect a surface of the access port of the vascular access device.

In some embodiments, the scrubbing cap comprises a seal attached over the cavity to seal the scrubbing insert within the cavity.

In some embodiments, the fluid connector component comprises a luer connector configured to be coupled to an access port or needleless connector of a vascular access device, and wherein the housing comprises a connector portion configured to engage with the luer connector to secure the scrubbing cap to the syringe assembly.

In some embodiments, the luer connector comprises one of a slip luer, a threaded luer, or a luer lock with collar.

In some embodiments, the housing comprises a connector portion configured to secure the scrubbing cap to the fluid connector component via a twist-type engagement, and the scrubbing cap is removable to enable connection of the syringe assembly to the access port or needleless connector of the vascular access device.

In some embodiments, the fluid access component comprises a needle defining a lumen therein, and wherein the container comprises a cover pierceable by the needle to place the reservoir in fluid communication with the channel of the plunger assembly.

In some embodiments, the fluid access component comprises a female luer connector, and wherein the container comprises a male luer connector engageable with the female luer connector to place the reservoir in fluid communication with the channel of the plunger assembly.

In some embodiments, the fluid connector component comprises an opening formed therein and wherein the cannula is positioned within the opening, the cannula having a cross-section smaller than the opening.

In some embodiments, a gap between an outer diameter of the opening and the cannula forms a first fluid path from the fluid connector component into a fluid chamber defined by a distal end of the stopper and an inner surface of the distal end of the syringe, and wherein the cannula forms a second fluid path from the fluid connector component to the fluid access component.

Also provided herein is a syringe assembly including a syringe having a distal end and a proximal end, with the syringe defining a chamber and having a fluid connector component at the distal end. The syringe assembly also includes a plunger assembly including an adapter member, a receiving cavity, and a fluid access component. The adapter member has a distal end including a stopper insertable into the proximal end of the syringe and slidable within the barrel, as well as a channel extending along a length thereof between the stopper and an opening of the receiving cavity. The fluid access component is positioned over the opening and extends into the receiving cavity. The syringe assembly further includes a cannula having a distal end and a proximal end, the distal end of the cannula secured within the fluid connector component, with the cannula extending proximally through the stopper to place the fluid connector component in fluid communication with the channel and the fluid access component. The syringe assembly still further includes a scrubbing cap secured to the fluid connector component of the syringe, the scrubbing cap including a housing secured to the fluid connector component and defining a cavity, and a scrubbing insert positioned within the cavity.

In some embodiments, the scrubbing insert comprises a resilient material including an antimicrobial solution or agent absorbed therein.

In some embodiments, the scrubbing cap comprises a seal attached over the cavity to seal the scrubbing insert within the cavity.

In some embodiments, the fluid connector component comprises a luer connector configured to be coupled to an access port or needleless connector of a vascular access device, and the housing comprises a connector portion configured to engage with the luer connector to secure the scrubbing cap to the syringe assembly.

In some embodiments, the connector portion of the housing secures the scrubbing cap via a twist-type engagement, and wherein the scrubbing cap is removable to enable connection of the syringe assembly to the access port or needleless connector of the vascular access device.

Also provided herein is a method of using a blood sample collection system. The method includes providing a syringe assembly including a syringe having a distal end and a proximal end, with the syringe defining a chamber and having a fluid connector component at the distal end. The syringe assembly also includes a plunger assembly including an adapter member, a receiving cavity, and a fluid access component. The adapter member has a distal end including a stopper insertable into the proximal end of the syringe and slidable within the barrel, as well as a channel extending along a length thereof between the stopper and an opening of the receiving cavity. The fluid access component is positioned over the opening and extends into the receiving cavity. The syringe assembly further includes a cannula having a distal end and a proximal end, the distal end of the cannula secured within the fluid connector component, with the cannula extending proximally through the stopper to place the fluid connector component in fluid communication with the channel and the fluid access component. The syringe assembly still further includes a scrubbing cap secured to the fluid connector component of the syringe, the scrubbing cap including a housing secured to the fluid connector component and defining a cavity, and a scrubbing insert positioned within the cavity. The method also includes positioning the scrubbing cap over an access port of a vascular access device, such that the proximal connector contacts the scrubbing insert, and cleaning the access port with the scrubbing insert.

In some embodiments, the fluid connector component comprises a luer connector configured to be coupled to the access port and the scrubbing cap comprises a connector portion engaged with the luer connector to secure the scrubbing cap to the syringe.

In some embodiments, the method further includes detaching the scrubbing cap from the luer connector subsequent to cleaning of the access port and coupling the luer connector to the access port, to place the syringe assembly in fluid connection with the vascular access device.

In some embodiments, the scrubbing cap comprises a seal attached over the cavity to seal the scrubbing insert within the cavity, and the method further comprises removing the seal from the scrubbing cap, to enable positioning of the scrubbing cap over the access port of the vascular access device and contacting of the scrubbing insert with the access port.

In some embodiments, cleaning the access port comprises applying a twisting motion to the blood sample collection system.

### BRIEF DESCRIPTION OF THE DRAWINGS

FIG. 1 is a side view of a blood sample collection system, including a syringe assembly and a collection container, according to an embodiment;

FIG. 2 is a detailed view of cut-out “A” of the syringe assembly of FIG. 1, according to an embodiment;

FIG. 3 is a front view of the syringe assembly of FIG. 1 with a scrubbing cap removed therefrom;

FIG. 4 is an exploded view of a scrubbing cap included in the blood sample collection system of FIG. 1, according to an embodiment;

FIGS. 5-8 are side views of the blood sample collection system of FIG. 1 illustrating process steps for using the system with an associated catheter assembly, according to an embodiment;

FIG. 9 is a detailed view of cut-out “B” of the syringe assembly and catheter assembly of FIG. 8, according to an embodiment; and

FIG. 10 is a side view of a syringe assembly, according to another embodiment.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw device in preparation for or during use. Similarly, in this application, the terms “in the distal direction” and “distally” mean in the direction toward an access connector portion of the fluid transfer device, and the terms “in the proximal direction” and “proximally” mean in the direction opposite the direction of the connector.

While not shown or described herein, it is to be understood that the blood sample collection systems described below may be utilized for blood draw from any suitable vascular access device (VAD) such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Embodiments of the present disclosure will primarily be described in the context of blood sample collection systems for use with peripheral IV catheters (PIVCs), midlines, PICCs, and central line catheters. However, embodiments of the present disclosure equally extend to use with other catheter devices.

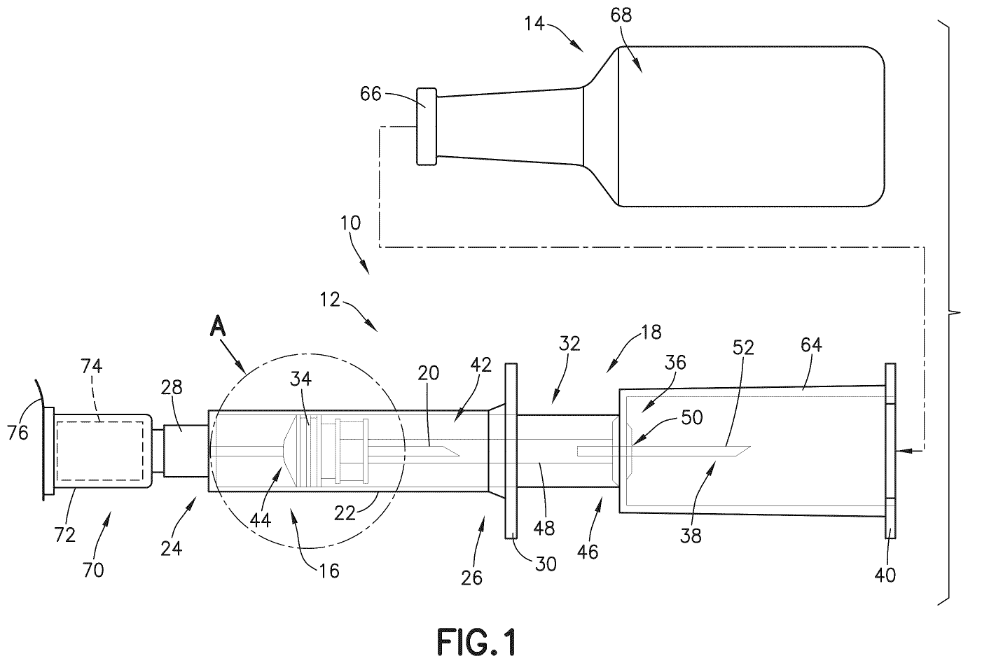

Referring to FIGS. 1-3, a blood sample collection system 10 is shown in accordance with an aspect of the present disclosure. The system 10 includes a syringe assembly 12 and a blood collection container 14 useable with the syringe assembly 12. As described in further detail below, the blood sample collection system 10 may be employed by a user (e.g., clinician) to first obtain an initial discard sample of blood in the syringe assembly 12 and then subsequently obtain a blood culture sample in the collection container 14 that may be analyzed to detect the presence of bacteria or fungi.

As shown in FIG. 1, the syringe assembly 12 may be generally characterized as including a syringe 16, a plunger assembly 18, and a cannula 20. The syringe 16 may be structured as a generally cylindrical barrel 22 that includes a distal end 24 and a proximal end 26, with a fluid connector component 28 at the distal end 24 and a finger grip 30 at the proximal end 26. According to embodiments, the fluid connector component 28 may be integrated with or removably coupled to the distal end 24 of the barrel 22. According to embodiments, the fluid connector component 28 may include any suitable component configured to couple the barrel 22 to a connector or access port of a vascular access device (VAD). For example, in some embodiments, the fluid connector component 28 may be a luer connector. The luer connector may be in the form of, e.g., a slip luer, a threaded luer, a luer lock with collar, etc. In some embodiments, the fluid connector component 28 can be an outlet of the barrel 22 defining a lumen.

The plunger assembly 18 includes an adapter member 32, a stopper 34, a receiving cavity 36, a fluid access component 38, and a finger grip 40. In one embodiment, the adapter member 32, receiving cavity 34, and finger grip 40 are a single integral component. For example, the adapter member 32, receiving cavity 34 and finger grip 40 may be formed as a molded component. Alternatively, the adapter member 32, receiving cavity 34 and finger grip 40 may be fabricated individually and thereafter connected to one another.

As shown in FIG. 1, adapter member 32 is formed as a generally elongated member that is insertable into a chamber 42 defined by the barrel 22 of syringe 16, with the adapter member 32 having a distal end 44 and a proximal end 46. A channel 48 is formed within the adapter member 32 extending the length thereof. In one embodiment, the channel 48 is tapered along its length—with a diameter of the channel 48 increasing as the channel 48 extends from the distal end 44 to the proximal end 46. The proximal end 46 of the channel 48 includes an opening 50 thereat that transitions into receiving cavity 36.

In accordance with aspects of the disclosure, the fluid access component 38 is positioned within the opening 50, at the proximal end 46 of channel 48. The fluid access component 38 may be secured within opening 50 and extend out from the proximal end 46 of channel 48 into the interior of the receiving cavity 36. In one embodiment, the fluid access component 38 may include a needle 52 defining a lumen therein. In some embodiments, the fluid access component 38 may include a flexible needle sheath (not shown) configured to substantially surround and be translated relative to the needle 52 such that a proximal end of the needle 52 may be selectively exposed.

The stopper 34 of plunger assembly 18 is positioned at the distal end 44 of adapter member 32 so as to be movable along with the adapter member 32 within the chamber 42 of syringe barrel 22. The stopper 34 may be made from a material that is different from the material of the adapter member 32 and that is capable of forming a tight seal with the syringe barrel 22 as it is advanced therethrough. In some embodiments, the stopper 34 includes a receptacle (not shown) formed therein that is sized and configured to receive a flanged extension member 50 formed on the distal end 44 of adapter member 32, with the flanged extension member coupling with the receptacle via a press fit connection, for example, to secure the stopper 34 to the adapter member 32, although it can be appreciated that the stopper 34 can be secured to the distal end 44 of adapter member 32 by other techniques known in the art.

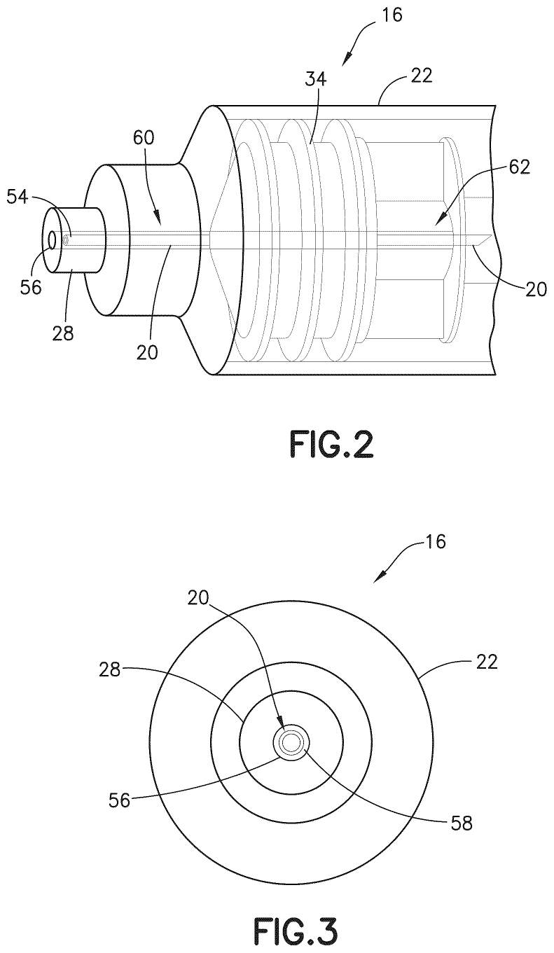

Referring still to FIGS. 1-3, it is shown that cannula 20 is incorporated into syringe assembly 12 via positioning thereof within syringe 16 and plunger assembly 18. The cannula 20 is provided in syringe assembly 12 such that a distal end 54 thereof is positioned substantially flush with an opening 56 in fluid connector component 28 of syringe 16, with the cannula 20 extending proximally from the fluid connector component 28, through a portion of chamber 42 of barrel 22 and through a hole formed in stopper 34, and into the channel 48 formed in adapter member 32, as best shown in FIG. 2. In some embodiments, the cannula 20 may be recessed rearward of the opening 56 in the fluid connector component 28, with this recessed arrangement providing the syringe assembly 12 compatibility with needleless connectors, luer-activated valves and/or IV connectors that may have an internal spike or similar projection intended to insert into the fluid connector component 28 for functionality.

As shown in FIG. 3, the cannula 20 has a cross-section smaller than the opening 56 at the tip of fluid connector component 28, allowing fluid to flow through a gap 58 between the outer surface of the cannula 20 and the inner surface of the opening 56. According to embodiments, the cannula 20 is secured to fluid connector component 28 so as to not impede the flow of fluids between the gap 58. In one embodiment, the cannula 20 is secured to the fluid connector component 28 via one or more retaining features of the fluid connector component 28, which may comprise flanges, struts, wings, a beveled flange, or other mechanical structures (not shown) extending between the inner surface of the fluid connector component 28 and the outer surface of the cannula 20. In other embodiments, the cannula 20 may be secured to the fluid connector component 28 using an adhesive, thermal bonding, or other suitable means.

With cannula 20 sized and positioned relative to syringe 16 and plunger assembly 18 as described above, separate fluid paths are provided in syringe assembly 12 via which blood may be drawn into system 10. A (first) fluid path 60 is formed via the gap 58 between the outer surface of the cannula 20 and the inner surface of the opening 56 through which fluid (i.e., blood) may flow from fluid connector component 28 and into a portion of chamber 42 within barrel 22—i.e., a fluid chamber defined by a distal surface of the stopper 34 and an inner surface of the distal end 24 of barrel 22. A (second) fluid path 62 is provided via the cannula 20 through which fluid (i.e., blood) may flow from fluid connector component 28 and into the channel 48 of adapter member 32, with the fluid then being transferrable out from channel 48 and through fluid access component 38 into a container 14.

Referring again to FIG. 1, the container 14 may have any of a number of structures that enable the container 14 to be inserted into receiving cavity 36 (defined by a cylindrical wall structure 64), with FIG. 1 illustrating container 14 as a necked vial, but it recognized that container 14 could be provided as a tube or other suitable receptacle, such as a BD VACUTAINER® from Becton, Dickinson and Co., as a non-limiting example. The container 14 includes include a cover 66 and a fluid reservoir 68, with the cover 66 disposed at a distal end of the container 14. In some embodiments, the cover 66 may include a resealable membrane configured such that a fluid access component, such as fluid access component 38, can pierce the resealable membrane to achieve fluidic communication with the reservoir 68. The resealable membrane of the cover 66 may be configured to reseal upon decoupling the fluid access component 38 from the cover 66 such that the reservoir 68 is fluidically isolated from an area external to the container 14. The reservoir 68 can be an evacuated reservoir such that, upon the reservoir 68 being placed in fluidic communication with a source of fluid (e.g., via piercing the resealable membrane of the cover 66 with a fluid access component fluidically coupled to a patient's vasculature), fluid (e.g., blood) can be drawn into the reservoir 68 due to a pressure differential between the reservoir 68 and the source of fluid.

According to aspects of the disclosure, it is recognized that when initially connecting the blood sample collection system 10 to a VAD access port, such as via coupling of the fluid connector component 28 with the access port, it is desirable to disinfect the access port prior to connection of blood sample collection system 10 thereto. That is, it is desirable to clean and sterilize the access port prior to engagement of fluid connector component 28 therewith (e.g., via mating of female and male luer connections) to prevent microbial ingress and possible catheter-related blood stream infections (CRBSIs).

According to aspects of the disclosure, a scrubbing cap 70 is integrated in with the syringe assembly 12 to provide for cleaning and sterilizing of the access port prior to engagement of the system 10 therewith. The scrubbing cap 70 is secured onto fluid connector component 28 and is configured to engage with an access port to enable the cleaning and sterilizing of internal and/or external surfaces thereof, such as via a twisting and scrub motion between the scrubbing cap 70 and access port. The scrubbing cap 70 may be removed from fluid connector component 28 upon completion of the cleaning and sterilizing of the access port, thereby enabling a subsequent coupling of fluid connector component 28 to the access port and performing of a blood draw via operation of system 10 as previously described.

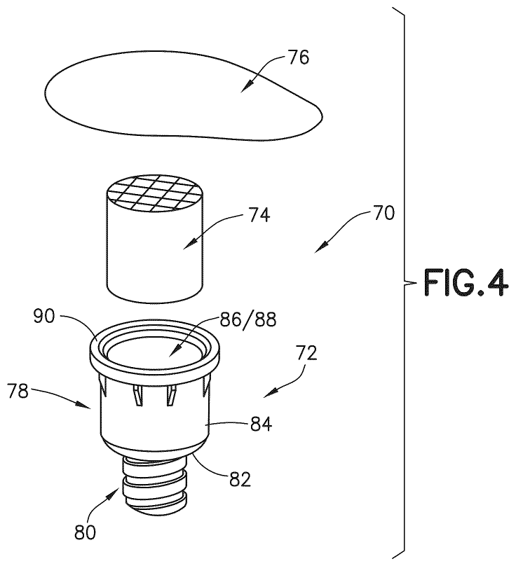

As shown in FIGS. 1 and 2 and now also in FIGS. 4 and 5, scrubbing cap 70 includes a cap housing 72, scrubbing insert 74, and a pealable seal 76. The housing 72 may be an integrally formed structure made by injection molding and may be made of an alcohol compatible material, such as polypropylene or polyethylene, for instance. The housing 72 may generally be described as including a holder portion 78 that retains the insert 74 therein and a connector portion 80 configured to mate with the fluid connector component 28 of blood sample collection system 10.

The holder portion 78 may have a cup shape that is formed by a base 82 and a sidewall structure 84 that defines an opening 86 on one end thereof. The sidewall structure 84 is formed integrally with the base 82 to define a cavity 88 configured to receive the insert 74 therein. In some embodiments, the cavity 88 may have a cylindrical cross-sectional shape and may have a depth suitable to receive the insert 74 therein in a recessed manner. However, it is appreciated that the holder portion 78 and the cavity 88 defined thereby can assume other shapes, including square, hexagonal, etc., and that the holder portion 78, its cavity 88, and the insert 74 disposed therein can be configured in shape and size so as to enable the scrubbing cap 70 to cleanse a particular size and configuration of a medical device with which the scrubbing cap 70 is to be employed (i.e., catheter connector 20 of catheter assembly 12).

The holder portion 78 may be sized such that the cavity 88 defined by sidewall structure 84 compresses the insert 74 when the insert 74 is fitted into the cavity 88, so as to retain the insert 74 therein. A suitable hot melt glue or other suitable adhesive may also be used to adhere the insert 74 to the bottom of the holder portion 78 (i.e., to base 82), although it is recognized that other suitable methods can also be employed to secure the insert 74 to the holder portion 78, including mechanical fixation for instance. An annular lip 90 may be formed on the holder portion 78 (i.e., on sidewall structure 84) about opening 86 to define a land for receiving the seal 76, with the seal 76 cooperating with the annular lip 90 to seal the opening 86 of the cavity 88 and retain the insert 74 therein. The seal 76 seals the cavity 88 of the holder portion 78 and the insert 74 therein against contamination from the outside environment and provides a leak-proof barrier, thereby protecting the contents of insert 74 and maintaining a sealed, sterilized environment. The seal 76 provides a sufficient seal at a range of temperatures, pressures, and humidity levels and, according to embodiments, may be formed as an aluminum or multi-layer polymer film peel back top. In some embodiments, the seal 76 is heat-sealed or induction sealed to the open end of the scrubbing cap 70. The seal 76 can include a tab 92 to facilitate the manipulation of and removal of the seal 76 from the scrubbing cap 70.

The insert 74 is constructed of a foam material, for example, of injection molded construction or the insert 74 may be die-cut from a foam sheet. The insert includes a cleansing substance impregnated therein (while in the holder portion 78), such as a solution of a suitable microbiocide or germicide. The cleansing substance can include an anti-bacterial disinfectant of any suitable type and suitable amount depending upon the size of the insert of foam material. For example, in one embodiment use is made of an aqueous solution including about two percent (2%) chlorhexidine gluconate (chlorhexidine solution, “CHG”) by volume in an amount of from about 0.20 cc to about 0.75 cc. Optionally, a solution including about 0.50 cc is employed. In another embodiment, a solution including about 70 percent (70%) isopropyl alcohol (“IPA”) in an aqueous solution is included in the cleansing substance. In yet another embodiment, a solution including about 70 percent (70%) IPA and about two percent (2%) CHG in an aqueous solution in an amount of about 0.2 ml is included in the cleansing substance. In the latter solution, it is recognized that the concentration of IPA can vary from about 60 percent (60%) to about 90 percent (90%) and the concentration of CHG can vary from about one percent (1%) to about five percent (5%), in one embodiment. Other suitable solution compositions and concentrations are also possible. For instance, povidone iodine or hydrogen peroxide solutions can be included in the cleansing substance, in one embodiment.

According to aspects of the disclosure, the insert 74 may be constructed (e.g., molded) to have a predefined shape that conforms to the unique shape of the VAD access port or connector that is to be cleaned thereby. The insert 74 may further have any of a number of constructions that provide for effective cleaning of the port/connector, such as including a patterned or roughened top surface and/or gaps or slits formed in the foam material thereof that enables the insert 74 to better deform/conform about interior and exterior surfaces of the port/connector.

The connector portion 80 of scrubbing cap 70 extends proximally away from holder portion 78 and is configured to mate with fluid connector component 28 to secure the scrubbing cap 70 thereto. According to embodiments, the connector portion 80 may be configured as a luer connection (e.g., male luer connection or female luer connection) that enables mating thereof with the corresponding luer connection provide by fluid connector component 28, such that the connector portion 80 is secured in place relative to fluid connector component 28.

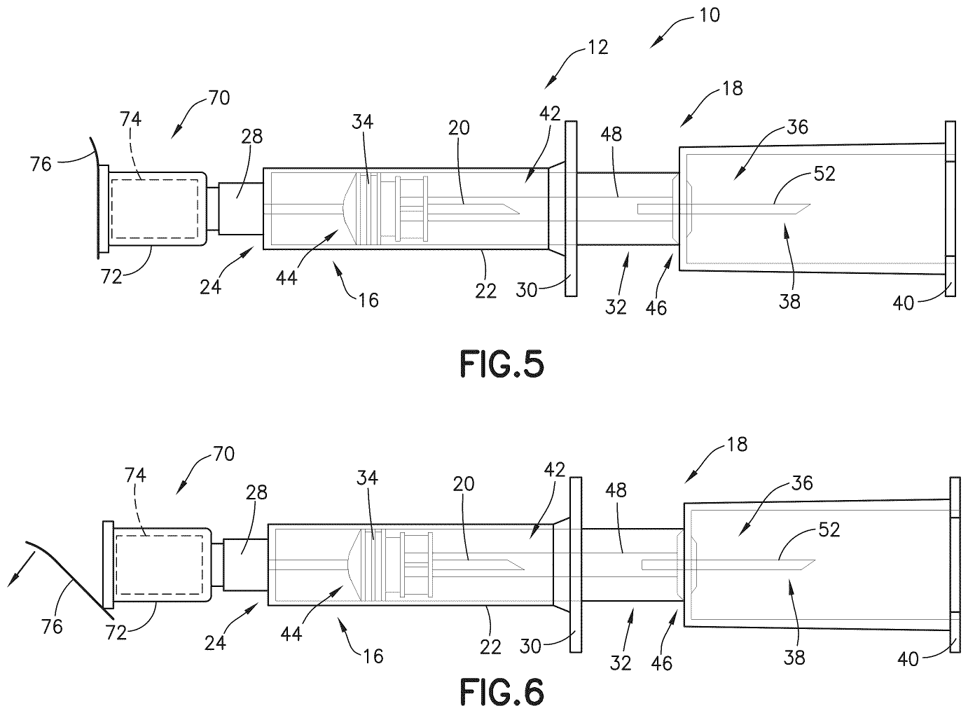

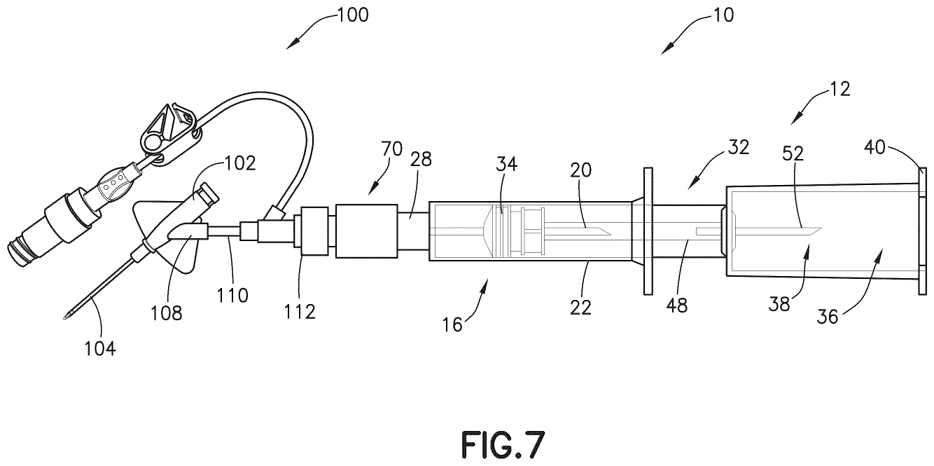

Following here below, and as shown in FIGS. 5-8, use of the system 10 is described in accordance with one aspect of the disclosure. Use of the system 10 begins with the syringe assembly 12 in an initial configuration as shown in FIG. 5—where scrubbing cap 70 is secured to the fluid connector component 28 of syringe 16 and the scrubbing cap 70 is in a sealed condition (i.e., seal 76 is secured to housing 72, so as to seal-off scrubbing insert 74). Use of the system 10 continues by removing the seal 76 from the scrubbing cap 70 of syringe assembly 12 in order to provide access to the scrubbing insert 74, as shown in FIG. 6. After removal of the seal 76, the syringe assembly 12 is brought into proximity to a catheter assembly 100 to provide for cleaning and sterilization of an access port thereto. In the non-limiting embodiment illustrated in FIG. 7, catheter assembly 100 includes a catheter adapter 102 and associated catheter 104 (that may be inserted into the vasculature of a patient), as well as a connector 106 coupled to an adapter port 108 on the catheter adapter 102 (such as via a fluid conduit 110). Connector 106 may be a t-connector (e.g., one side port arranged at a 90 degree angle relative to a longitudinal axis of connector 106), a y-connector (e.g., one side port arranged at a 25, a 60, or a 75 degree angle relative to a longitudinal axis of connector 106), or any other type of connector known in the art. In some embodiments or aspects, connector 106 may have a luer connector or needleless access connector 112 coupled to the proximal end thereof, with the needleless access connector 112 providing a near-patient access port to the catheter assembly 100.

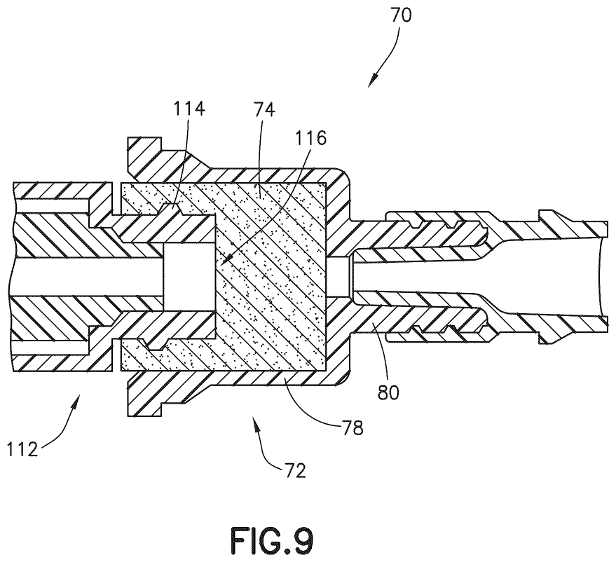

Upon the syringe assembly 12 brought into proximity to the catheter assembly 100, as shown in FIG. 7, the scrubbing cap 70 is positioned over the connector 112 (which may be configured as a female luer connection having a threaded outer connection 114 defining a tapered lumen or cavity 116, see FIG. 9, according to a non-limiting embodiment), with the foamed insert 74 pushed onto the connector 112 by the user. At this time, portions of the insert 74 conform about and/or are inserted into the connector 112 (i.e., about threaded outer connection 114 and into lumen 116). Once the portion of the connector 112 has been inserted into the foamed insert 74 of the scrubbing cap 70, the scrubbing cap 70 is rotated relative to the connector 112. For example, the user may hold the connector 112 stationary while rotating the blood sample collection system 10. As the scrubbing cap 70 is secured in place relative to the blood sample collection system 10—via the connection of fluid connector component 28 to scrubbing cap 70—rotation of the blood sample collection system 10 causes a corresponding rotation of scrubbing cap 70. Rotation of the scrubbing cap 70 relative to the connector 112 may be done all in one direction or may be a back-and-forth twisting motion. The scrubbing cap 70 is rotated a sufficient number of times relative to the connector 112 to sufficiently kill any bacteria that the solution-impregnated foam insert 74 comes in contact with and/or to remove any biofilm from the outside peripheral surface and threaded outer connection 114 of connector 112 as well as the inside surface of the lumen 116 of the connector 112. In this way, both exterior surfaces and interior luminal surfaces of the connector 112 are scrubbed by the insert 74, causing the cleansing substance therein to disinfect the surfaces and remove any biofilm disposed thereon.

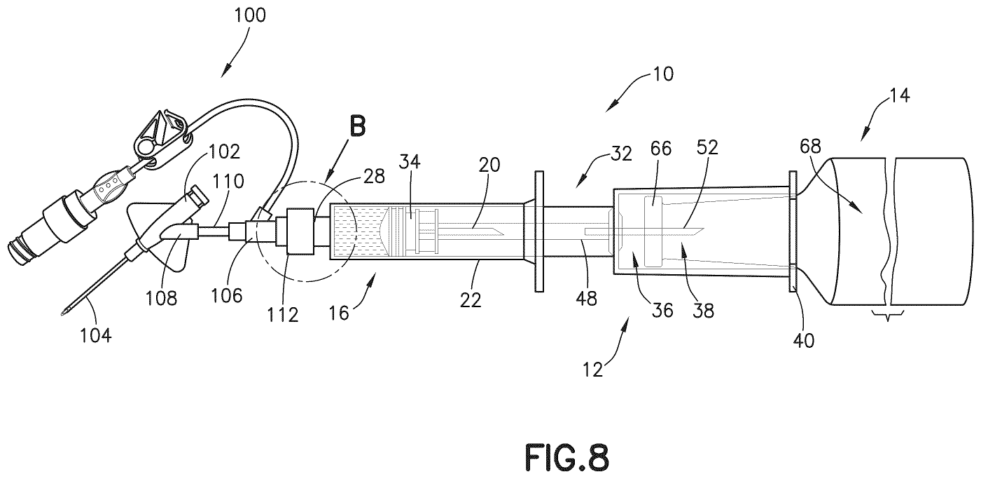

Upon completion of the cleaning and sterilizing of the connector 112 of catheter assembly 100 via scrubbing cap 70, the scrubbing cap 70 is separated away from connector 112. The scrubbing cap 70 may then be removed from fluid connector component 28 of system 10. After allowing the connector 112 to dry, the blood sample collection system 10 may then be connected to the catheter assembly 100 by coupling the fluid connector component 28 to connector 112, as shown in FIG. 8, thereby connecting system 10 to catheter assembly 100.

According to an aspect of the disclosure, upon connection of blood sample collection system 10 to catheter assembly 100, an initial discard sample of blood may be drawn into syringe assembly 12. That is, the syringe assembly 12 may be actuated from an initial configuration to a diversion or discard configuration—with the plunger assembly 18 pulled back proximally relative to syringe 16, such that the stopper 34 moves proximally within syringe barrel 22. Upon the stopper 34 moving proximally within syringe barrel 22, a discard (or diversion) volume of blood is drawn into a portion of chamber 42—i.e., a fluid chamber defined by a distal surface of the stopper 34 and an inner surface of the distal end 24 of barrel 22—with blood flowing along a first fluid path (through gap 58) from fluid connector component 28 and into the portion of chamber 42, as shown in FIG. 8.

Upon drawing of a sufficient discard sample of blood, the blood sample collection system 10 may then subsequently be operated to acquire a desired blood culture sample within a collection container 14. For acquiring such a sample, a collection container 14 is engaged with syringe assembly 12—with the collection container 14 (e.g., vacutainer) positioned within the receiving cavity 36 and engaged with the fluid access component 38 to place the container 14 in fluid communication with the plunger assembly 18. That is, upon positioning/connection of the collection container 14 with receiving cavity 36 and fluid access component 38, a second fluid path is formed from the connector component 28 to the collection container 14—with cannula 20 transferring blood from connector component 28 into channel 48 within adapter member 32, and blood then passing from channel 48 through fluid access component 38 and into container 14. As described above, in some embodiments, fluid access component 38 is provided as a needle 52 with a lumen formed therein, with the needle 52 piercing the cover 66 of container 14 so that blood is drawn into the evacuated reservoir 68 within the container 14.

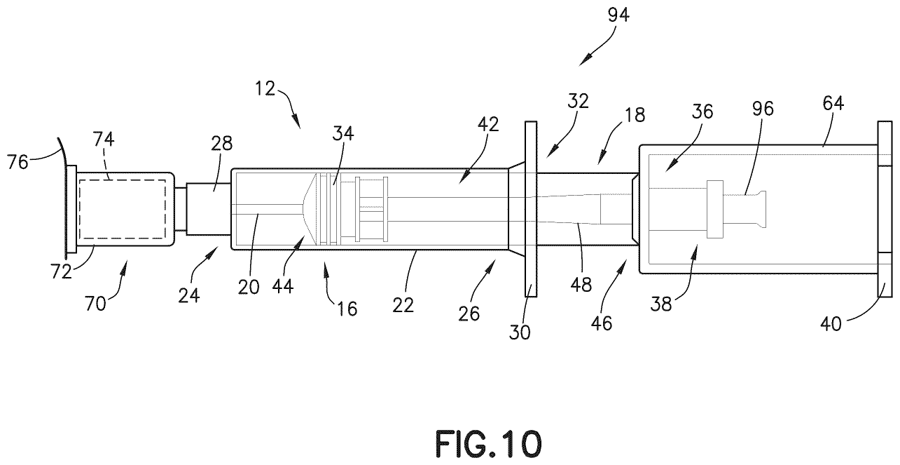

While blood sample collection system 10 is shown and described in FIGS. 1-8 according to a specific embodiment, it is recognized that the system 10 (and the syringe assembly 12 thereof) may have other suitable configurations. FIG. 10 illustrates a syringe assembly 94 in accordance with another aspect of the disclosure. The syringe assembly 94 is almost identical to the syringe assembly 12 previously shown and described in FIGS. 1-8, except that rather than fluid access component 38 being provided as a needle 52, fluid access component 38 is provided as a closed connector 96, such as a closed system female luer connector as provided in FIG. 10. The closed system female luer connector 96 is configured to receive and mate with an associated male luer connector of a collection container (not shown), in order to provide for collection of a blood sample within the collection container when the container is connected to the syringe assembly 94.

While several embodiments of a blood sample collection system configured for blood draw during catheter indwell were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is:

1. A blood collection system, comprising:
a syringe assembly comprising:
a syringe having a distal end and a proximal end, the syringe defining a chamber and a fluid connector component at the distal end;
a plunger assembly including an adapter member, a receiving cavity, and a fluid access component, the adapter member having a distal end including a stopper insertable into the proximal end of the syringe and slidable within the barrel, the adapter member including a channel extending along a length thereof between the stopper and an opening of the receiving cavity, with the fluid access component positioned over the opening and extending into the receiving cavity;
a cannula having a distal end and a proximal end, the distal end of the cannula secured within the fluid connector component, with the cannula extending proximally through the stopper to place the fluid connector component in fluid communication with the channel and the fluid access component; and
a scrubbing cap secured to the fluid connector component of the syringe, the scrubbing cap comprising:
a housing secured to the fluid connector component and defining a cavity; and
a scrubbing insert positioned within the cavity; and
a container defining a reservoir, the container positionable within the receiving cavity and engageable with the fluid access component to transfer fluid into the reservoir.

2. The system of claim 1, wherein the scrubbing insert comprises a resilient material including an antimicrobial solution or agent absorbed therein configured to disinfect a surface of the access port of the vascular access device.

3. The system of claim 2, wherein the scrubbing cap comprises a seal attached over the cavity to seal the scrubbing insert within the cavity.

4. The system of claim 1, wherein the fluid connector component comprises a luer connector configured to be coupled to an access port or needleless connector of a vascular access device, and wherein the housing comprises a connector portion configured to engage with the luer connector to secure the scrubbing cap to the syringe assembly.

5. The system of claim 4, wherein the luer connector comprises one of a slip luer, a threaded luer, or a luer lock with collar.

6. The system of claim 1, wherein the housing comprises a connector portion configured to secure the scrubbing cap to the fluid connector component via a twist-type engagement, and wherein the scrubbing cap is removable to enable connection of the syringe assembly to the access port or needleless connector of the vascular access device.

7. The system of claim 1, wherein the fluid access component comprises a needle defining a lumen therein, and wherein the container comprises a cover pierceable by the needle to place the reservoir in fluid communication with the channel of the plunger assembly.

8. The system of claim 1, wherein the fluid access component comprises a female luer connector, and wherein the container comprises a male luer connector engageable with the female luer connector to place the reservoir in fluid communication with the channel of the plunger assembly.

9. The system of claim 1, wherein the fluid connector component comprises an opening formed therein and wherein the cannula is positioned within the opening, the cannula having a cross-section smaller than the opening.

10. The system of claim 9, wherein a gap between an outer diameter of the opening and the cannula forms a first fluid path from the fluid connector component into a fluid chamber defined by a distal end of the stopper and an inner surface of the distal end of the syringe, and wherein the cannula forms a second fluid path from the fluid connector component to the fluid access component.

11. A syringe assembly comprising:
a syringe having a distal end and a proximal end, the syringe defining a chamber and a fluid connector component at the distal end;
a plunger assembly including an adapter member, a receiving cavity, and a fluid access component, the adapter member having a distal end including a stopper insertable into the proximal end of the syringe and slidable within the barrel, the adapter member including a channel extending along a length thereof between the stopper and an opening of the receiving cavity, with the fluid access component positioned over the opening and extending into the receiving cavity;
a cannula having a distal end and a proximal end, the distal end of the cannula secured within the fluid connector component, with the cannula extending proximally through the stopper and into the channel to place the fluid connector component in fluid communication with the fluid access component; and
a scrubbing cap secured to the fluid connector component of the syringe, the scrubbing cap comprising:
a housing secured to the fluid connector component and defining a cavity; and
a scrubbing insert positioned within the cavity.

12. The syringe assembly of claim 11, wherein the scrubbing insert comprises a resilient material including an antimicrobial solution or agent absorbed therein.

13. The syringe assembly of claim 11, wherein the scrubbing cap comprises a seal attached over the cavity to seal the scrubbing insert within the cavity.

14. The syringe assembly of claim 11, wherein the fluid connector component comprises a luer connector configured to be coupled to an access port or needleless connector of a vascular access device, and wherein the housing comprises a connector portion configured to engage with the luer connector to secure the scrubbing cap to the syringe assembly.

15. The syringe assembly of claim 14, wherein the connector portion of the housing secures the scrubbing cap via a twist-type engagement, and wherein the scrubbing cap is removable to enable connection of the syringe assembly to the access port or needleless connector of the vascular access device.

16. A method of using a blood sample collection system comprising:
providing a syringe assembly including:
a syringe having a distal end and a proximal end, the syringe defining a chamber and a fluid connector component at the distal end;
a plunger assembly including an adapter member, a receiving cavity, and a fluid access component, the adapter member having a distal end including a stopper insertable into the proximal end of the syringe and slidable within the barrel, the adapter member including a channel extending along a length thereof between the stopper and an opening of the receiving cavity, with the fluid access component positioned over the opening and extending into the receiving cavity;
a cannula having a distal end and a proximal end, the distal end of the cannula secured within the fluid connector component, with the cannula extending proximally through the stopper and into the channel to place the fluid connector component in fluid communication with the fluid access component; and
a scrubbing cap secured to the fluid connector component of the syringe, the scrubbing cap comprising:
a housing secured to the fluid connector component and defining a cavity; and
a scrubbing insert positioned within the cavity;
positioning the scrubbing cap over an access port of a vascular access device, such that the proximal connector contacts the scrubbing insert; and
cleaning the access port with the scrubbing insert.

17. The method of claim 16, wherein the fluid connector component comprises a luer connector configured to be coupled to the access port, and wherein the scrubbing cap comprises a connector portion engaged with the luer connector to secure the scrubbing cap to the syringe.

18. The method of claim 17, comprising:
detaching the scrubbing cap from the luer connector subsequent to cleaning of the access port; and
coupling the luer connector to the access port, to place the syringe assembly in fluid connection with the vascular access device.

19. The method of claim 16, wherein the scrubbing cap comprises a seal attached over the cavity to seal the scrubbing insert within the cavity, and wherein the method further comprises removing the seal from the scrubbing cap, to enable positioning of the scrubbing cap over the access port of the vascular access device and contacting of the scrubbing insert with the access port.

20. The method of claim 16, wherein cleaning the access port comprises applying a twisting motion to the blood sample collection system.
